(19) United States
(12) Patent Application Publication
(10) Pub. No.: US 2021/0022997 A1
(43) Pub. Date: Jan. 28, 2021

(54) Method of administering a Medication

(71) Applicant: Damn IP, LLC, Westlake, OH (US)

(72) Inventor: Darrin Farrow, Westlake, OH (US)

(73) Assignee: Damn IP, LLC, Westlake, OH (US)

(21) Appl. No.: 17/045,339

(22) PCT Filed: Apr. 3, 2019
(86) PCT No.: PCT/US19/25626

§ 371 Date: Oct. 5, 2020

Related U.S. Application Data
(60) Provisional application No. 62/652,529, filed on Apr. 4, 2018.

(51) Int. Cl. A61K 9/00; A61K 31/05; A61K 31/352; A61K 9/08; A61K 47/46

(52) U.S. Cl. CPC A61K 9/0053; A61K 31/05; A61K 47/46; A61K 9/08; A61K 31/352

(57) ABSTRACT

A method of treating a patient using a carrier/active substance mixture that is intended to gradually release a dosage of the active substance along the digestive tract to treat the digestive tract. Also disclosed is a method of making and marketing the mixture designed for such treatment in individual dose packaging. Further disclosed is a single dose sealed package of the mixture.

### CROSS-REFERENCES TO RELATED APPLICATIONS

This application is a national stage application of PCT application serial number PCT/US19/25626 filed on Apr. 3, 2019, which claims the benefit of U.S. provisional patent application Ser. No. 62/652,529 filed on Apr. 4, 2018, incorporated herein by reference.

### BACKGROUND

Medications and supplements (such as herbal formulations, drugs, nutrients, herbs, etc.) are often administered to patients orally. However, when administered in the typical manner, such medications/supplements may be quickly absorbed in the stomach or early parts of the small intestine. Time released formulations are available, but these may not be useful for situations where a drug or supplement is desired to be distributed along the walls of the intestines, for example.

It is known that in some cases, taking a drug dosage with a meal or another supplement high in fiber can help delay the absorption of the drug, but this approach relies on the patient remembering to take two different materials, the medications/supplements, and the fiber food or supplement, at the same time, and these substances may be undesirably separated in the digestive system during the digestive process, potentially defeating the purpose of the combination.

Hence, a method of administering medications/supplements that is better adapted to treating the walls of the most, or the entire, digestive track is desired.

### SUMMARY

Provided are a plurality of example embodiments, including, but not limited to, a method of delivering a medical, plant-based or other compound(s) blended with fiber or any consumable that has a slow digestive process by which the compounds are pressed against the digestive system and walls of the colon for direct absorption and treatment of the digestive system.

Also provided is a method of providing a treatment mixture for treating the digestive tract of a patient, comprising the steps of:
mixing an active substance with a carrier substance to form the treatment mixture;
packaging the treatment mixture for sale; and
dissolving the treatment mixture in a liquid for consumption by the patient, wherein subsequent to the consumption, the treatment mixture is configured to traverse a substantial portion of the digestive tract of the patient to treat the digestive tract with the active substance along the portion.

Further provided is a method of providing a treatment mixture for treating the digestive tract of a patient, comprising the steps of:
mixing an active substance with a carrier substance to form the treatment mixture;
packaging the treatment mixture into individual dose packages; and
treating the patient by administering an individual dose package to the patient, wherein subsequent to the administering step, the treatment mixture is configured to traverse a substantial portion of the digestive tract of the patient to treat the digestive tract with the active substance along the portion.

Also provided is a method of providing a treatment mixture for treating the digestive tract of a patient, comprising the steps of:
mixing cannabidiol with a soluble fiber to form the treatment mixture;
packaging the treatment mixture for sale;
dissolving the treatment mixture in a liquid for consumption by the patient, wherein subsequent to the consumption, the treatment mixture is configured to traverse a substantial portion of the digestive tract of the patient to treat the digestive tract with the active substance along the portion.

Further provided is a method of providing a treatment mixture for treating the digestive tract of a patient, comprising the steps of:
mixing cannabidiol with a soluble fiber to form the treatment mixture;
packaging the treatment mixture into individual dose packages; and
treating the patient by administering an individual dose package to the patient, wherein subsequent to the administering step, the treatment mixture is configured to traverse a substantial portion of the digestive tract of the patient to treat the digestive tract with the active substance along the portion.

Still further provided is a method of providing a treatment mixture for treating the digestive tract of a patient, comprising the steps of:
mixing cannabidiol with a soluble fiber to form the treatment mixture; and
packaging the treatment mixture into individual dose packages.

Further provided is a method of providing a treatment mixture for treating the digestive tract of a patient, comprising the steps of:
mixing cannabidiol with a soluble fiber to form the treatment mixture;
packaging the treatment mixture into individual dose packages;
packaging a plurality of the individual dose packets into a package for retail sale, wherein
the treatment mixture is configured to traverse a substantial portion of the digestive tract of the patient to treat the walls of the digestive tract with the active substance along the portion.

Also provided is a method of providing a treatment mixture for treating the digestive tract of a patient, comprising the steps of:
mixing a plurality of different cannabinoids with a soluble fiber to form the treatment mixture;
packaging the treatment mixture for sale; and
configuring the treatment mixture for dissolving in a liquid for consumption by the patient so that said treatment mixture is configured to traverse a substantial portion of the digestive tract of the patient subsequent to said consumption to treat the digestive tract with the active substance along said portion.

Still further provided is a package providing a treatment mixture for treating the digestive tract of a patient, comprising: cannabidiol of an amount providing a single dosage; a soluble fiber mixed with the cannabidiol; and a sealed enclosure enclosing the single dose of cannabidiol mixed with the fiber sealed therein.

Also provided are additional example embodiments, some, but not all of which, are described hereinbelow in more detail.

### BRIEF DESCRIPTION OF THE DRAWINGS

The features and advantages of the example embodiments described herein will become apparent to those skilled in the art to which this disclosure relates upon reading the following description, with reference to the accompanying drawings, in which:

FIG. 1 is a flow chart of an example treatment process using an example mixture as provided herein;

FIG. 2 is a flow chart of a mixing process for creating an example mixture for use in the example treatment provided in FIG. 1; and

FIG. 3 is a drawing of an example single-dose packet that can be used for distributing the example mixture as provided in FIG. 2.

### DETAILED DESCRIPTION OF THE EXAMPLE EMBODIMENTS

Disclosed is a method of treating a patient using a fiber/medication mixture that is intended to gradually release a dosage of medication along the digestive tract. Also disclosed is a method of making and marketing the mixture designed for ease of use for such treatment.

To practice one of the methods of administering a medication/supplement (i.e., an active substance) where the active substance is released along a substantial portion of the digestive (or intestinal) tract, or even the entire digestive (or intestinal) tract, a medication/supplement is combined with a food supplement containing an undigestible, or slowly digested, fiber as a carrier. The fiber is used to ensure the passage of the active substance through a substantial portion of the digestive tract, in some cases slowing the passage and/or absorption rate, and by act of the fiber/carrier swelling and expanding, putting the active substance into actual contact with the walls and other structures of the digestive tract along its length. This ensures that the active substance, which may be a medication or health supplement, can act directly on the digestive organs for treating those organs, for example.

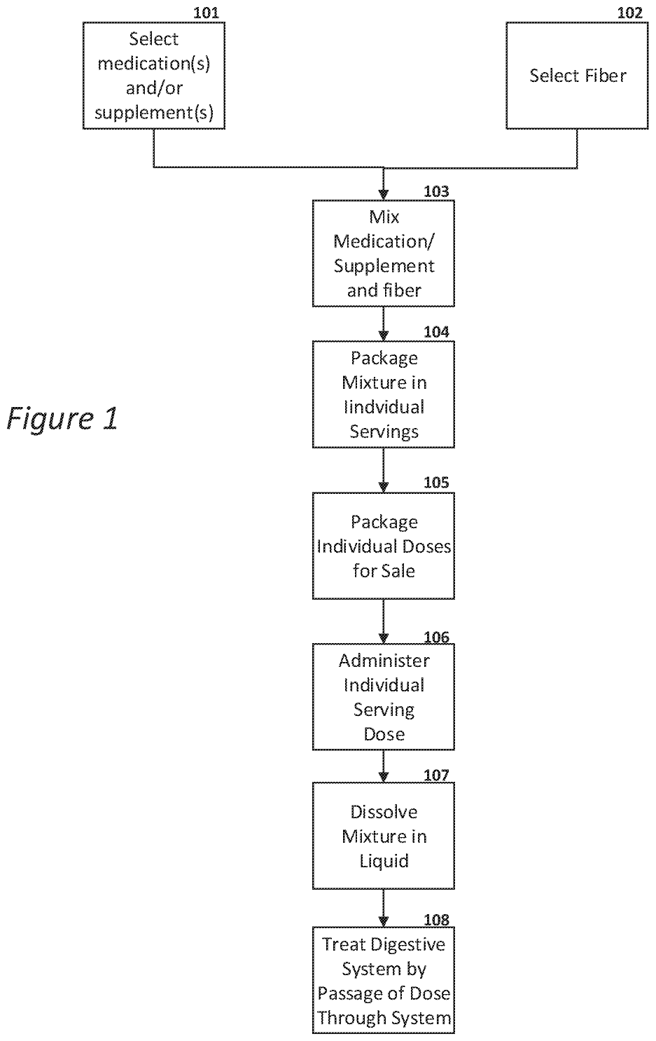

FIG. 1 shows a flow chart of an example method of treatment. The medication/supplement is selected 101 based on the type of treatment desired. For example, an anti-inflammatory agent, such as cannabidiol (CBD), can be selected to treat the digestive tract for disorders that result from, or result in, inflammation. Other substances such as THC and/or other cannabinoids can also be added to treat the digestive tract, and/or provide pain relief or other beneficial effects. Other substances such as nutrients, vitamins, and/or herbs with active components can also be added. The fiber helps delay the absorption of those substances as well.

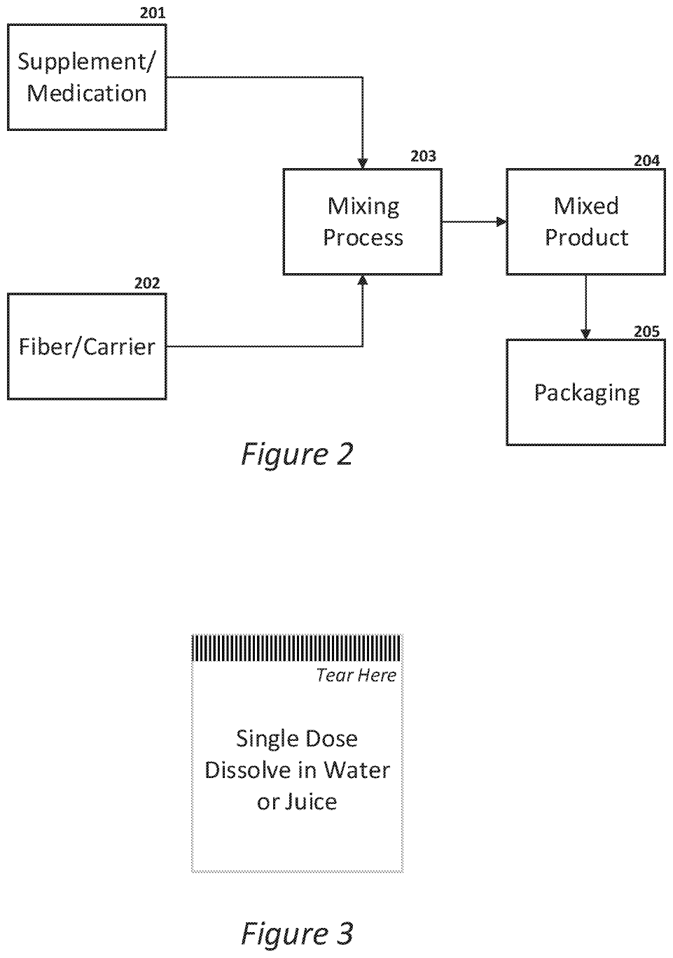

Referring still to the FIG. 1 flow chart, an appropriate fiber carrier (e.g., soluble fiber) is then selected 102, and then mixed with the medication/supplement 103 to form a mixture which can then be packaged. FIG. 2 shows this process of mixing 103 a supplement/medication 201 with a fiber/carrier 202 to form a mixed product 204, which can then be packaged 205, as desired.

Returning to the method of treatment shown in FIG. 1, the mixture can be packaged in individual servings 104 in order to make distribution and use as simple as possible. FIG. 3 shows an example of such an individual dose package including a single serving, where the mixture is packaged in a small pouch, such as a foil, paper, plastic, or other pouch that is perforated for ease of opening.

Returning to FIG. 1, the individual packages can be packaged for sale 105, such as in a box or other package containing a plurality of individual doses. Of course, an alternative is to package the mixture in bulk such as in a canister, allowing the user to measure out a dose as desired, such as by measuring with a teaspoon or tablespoon, or a measuring scoop that can be provide in the packaging. In any case, the user administers an individual dose 106 such as by dissolving the mixture with a liquid such as water or juice 107, thereby treating the digestive system as the mixture passes through the digestive system 108.

This treatment process can be used by a doctor for medical treatment, or as a homeopathic remedy not under direction of a doctor, but perhaps under the direction of a nutritionist or other professional, or self-administered.

Commercially available fiber supplements that are available to act as a carrier for the medication/supplement can include food-grade fibers, such as may be found in supplements like Metamucil®, which contain a non-digestible fiber such as psyllium fiber, or Citrucel®, which contains a methylcellulose fiber. Other types of fiber supplements may include soluble or insoluble fibers and/or brans obtained from wheat, oats, guar gum, polydextrose, resistant starch, glucomannan, or other food or even non-food items such as cellulose. Note that even soluble fibers tend to pass through the small intestine and much of the large intestine, where they may be metabolized by naturally occurring gut bacteria. In this way, even the large intestine may be treated with the desired active substances using this methodology.

Alternatively, any substance that may swell and slow absorption through the digestive tract and that can ensure that the active substance(s) (the medication and/or supplement) are put into physical contact with the walls of the digestive system could be used in place of the fiber, including digestible or undigestible materials such as oils or brans, for example.

In order to control the dosage of the medication/supplement to an appropriate amount, a desired amount of the medication/supplement is obtained in a form that can be mixed with a desired amount of the fiber, which is preferably in a powdered form. The medication/supplement may also be in powdered form, or even in some cases in a liquid form, which is then added to the fiber and dried to form the soluble mixture. The medication/supplement amount is well mixed with an appropriate amount of the chosen fiber carrier, such as a single recommended nutritive dose of the fiber. If the medication/supplement was in liquid form, after mixing the mixture may need to be dried and then pulverized back into a powder, if desired. In some cases, a liquid such as water may be used to mix the materials together, in particular where the desired end result may be in pill form which may then be administered by washing down the pill with a liquid such as water. A juice or other liquid could be use in place of the water.

Once mixed, the mixture should be packaged in an appropriate way, along with instructions for use. For example, if a powdered mixture is desired, the mixed material may be separated into individual dosages, which are then packaged in individual, single serve packets, such as small sealed packets (similar to the way individual serve sweeteners such as sugar or Equal® are packaged, for example), such as described above regarding FIG. 1, and with the packaging as shown in FIG. 3. The packaging may be made waterproof to keep the powder from solidifying, such as by using a metallic foil packaging or mylar.

Note that in some cases, larger amounts of the medication/supplement and fiber can be combined so that the resulting powdered mixture is packaged in a larger container, such that measured dosages can be used by the user similar to the manner in which the fiber is administered. Such dosages may be in the amount of a normal measuring spoon, such as a teaspoon or tablespoon, or a scoop holding an appropriate dosage amount may be included in the package. However, since separation may occur if the substances are of different particle weights, this approach may be of limited practicality for some mixtures with the single dosage packets being preferred.

In either case, the medication/supplement and fiber mixture can be taken in a normal manner used for fiber supplements, such as by dissolving the mixture into a liquid such as water or juice, and drinking it.

As an alternative approach, the medication/supplement and fiber mixture may be compressed into a pill or provided into a capsule or tablet, using techniques known in the medication and supplement industries, which are then taken orally in the normal manner, which will then dissolve in the stomach and travers the intestinal tract as desired.

Another approach is that the medication/supplement is physically or chemically bound to the fiber using a process where the medication/supplement is absorbed or chemically bound to the individual fiber particles in a manner where they are separated by the digestive process in the intestinal tract, or by dissolving in digestive fluids.

A further approach would be to combine the medication/supplement and fiber into a food item, such as a bar of food that is consumed by the user, providing the appropriate dosage of the medication/supplement in a single serving of the food item.

The individual dosages, whether provided as powders in individual packages, or in pill or capsule form, can be further packaged into pill bottles or multi-packs, as desired, and sold on the retail market without prescription, or sold by pharmacies with a doctor's prescription, as appropriate for the particular medication/supplement being sold.

Once the dosage is taken or consumed, the fiber slows the absorption of the medication/supplement so that it is slowly distributed along the digestive tract. This is particularly useful for medications/supplements that are intended to treat the digestive tract itself, such as by interacting with the walls of the intestine, for example.

Hence, the medication/supplement is configured to traverse the digestive tract so that it travels along a portion, or the entire, digestive tract along with the fiber. Due to the bulk and movement of the fiber, the medication/supplement comes into contact with the walls of the digestive tract for absorption by, or surface treatment of, the digestive tract itself. By increasing the amount of fiber mixed with the medication/supplement, doses of the medication/supplement can be administered at individual points along longer and longer stretches of the digestive track, thereby treating a substantial portion, or all, of the digestive tract with the active substance.

Note that this process may also be used to administer combinations of multiple medications and/or supplements by combination with the fiber in a single dosage mixture.

This process is particularly useful for treating disorders and diseases of the digestive system, such as inflammatory or immune diseases such as celiac disease, irritable bowel syndrome, Crohn's disease, colitis, diverticulitis, ulcers, or treatments for infections, tumors, or cancers, for example. Medications or supplements such as anti-inflammatories (such as turmeric), vitamins, pain killers, probiotics, and other treatments can be administered along the intestines using this process, ensuring that the medication/supplement is administered along substantial portions of the digestive tract, including even, in some cases, the large intestine since fiber travels along the entire tract.

One example embodiment is used to administer cannabidiol (CBD), a non psychoactive, non-euphoric, anti-inflammatory constituent of marijuana (or hemp or even certain other related plants), to the intestines. The use of CBD in treatment of digestive disorders, in particular those related to inflammation, has proven useful, and CBD has demonstrable neuroprotective and neurogenic effects, and even anti-cancer properties are currently being investigated. Because CBD is considered an herbal supplement, it can be used without a medical prescription. Since CBD can be obtained from plants (other than marijuana) that can be legally grown in most jurisdictions, the supplement can be legally marketed in most jurisdictions, including the United States.

In some situations, THC and/or other cannabinoids, such as may be derived from marijuana (including CBD and various terpenes) may be added to the mixture as additional active substances. These additional substances might add pain relief, and can provide some counteraction to the side effects of CBD. Note also that CBD (and other cannabinoids) helps counteract side effects of THC, so there are many instances where such combinations can be used for effectively treating various disorders.

For one embodiment of application of CBD to the intestines, a powdered formulation containing CBD is mixed with a powdered fiber to be packaged into individual dosage packets, which are sold on the herbal supplement market, using a process such as described above regarding FIGS. 1-3. The packaged dosage can be dissolved in a liquid such as water or juice, which is then consumed by the patient. Such consumption may be recommended to occur on an empty stomach without food, or in some cases may be taken with food. The CBD (and any additional active substances that might also be added to the mixture) then flows along the digestive tract, including the intestinal tract, along with the combined fiber, being administered to a large portion of the digestive tract, and thereby treating the digestive tract with the desired active substance leading to the desired effect, such as a reduction in inflammation among other possible desired affects.

Other anti-inflammatory supplements might be utilized in addition to, or instead of, CBD, such as turmeric, vitamins, herbs, Omega 3 oils, aspirin, acetaminophen, naproxen, ibuprofen, opiods, terpenes, THC, other cannabinoids, or other supplements or medications, for example. For example, an anti-inflammatory may be combined with a pain reliever or nutritional supplement. Any combinations of these substances, when mixed with the fiber, can result in delayed absorption and treatment of the intestinal tract.

Many other example embodiments can be provided through various combinations of the above described features. Although the embodiments described hereinabove use specific examples and alternatives, it will be understood by those skilled in the art that various additional alternatives may be used and equivalents may be substituted for elements and/or steps described herein, without necessarily deviating from the intended scope of the application. Modifications may be necessary to adapt the embodiments to a particular situation or to particular needs without departing from the intended scope of the application. It is intended that the application not be limited to the particular example implementations and example embodiments described herein, but that the claims be given their broadest reasonable interpretation to cover all novel and non-obvious embodiments, literal or equivalent, disclosed or not, covered thereby.

1. A method of providing a treatment mixture for treating the digestive tract of a patient, comprising the steps of:
mixing an active substance with a carrier substance to form the treatment mixture;
packaging the treatment mixture for sale; and
dissolving the treatment mixture in a liquid for consumption by the patient, wherein subsequent to said consumption, said treatment mixture is configured to traverse a substantial portion of the digestive tract of the patient to treat the digestive tract with the active substance along said portion.

2. The method of claim 1, wherein said carrier is a soluble fiber.

3. The method of claim 1, wherein said packaging includes the step of packaging said treatment mixture into individual dose packages.

4. The method of claim 1, wherein said active substance is an anti-inflammatory agent.

5. The method of claim 1, wherein said active substance is cannabidiol.

6. The method of claim 1, wherein said treatment mixture is configured to treat the walls of the digestive tract during traversal of the digestive tract.

7. The method of claim 1, wherein said active substance includes cannabidiol and THC.

8. A method of providing a treatment mixture for treating the digestive tract of a patient, comprising the steps of:
mixing an active substance with a carrier substance to form the treatment mixture;
packaging the treatment mixture into individual dose packages; and
treating the patient by administering an individual dose package to the patient, wherein subsequent to said administering step, said treatment mixture is configured to traverse a substantial portion of the digestive tract of the patient to treat the digestive tract with the active substance along said portion.

9. The method of claim 8, wherein said carrier is a soluble fiber.

10. The method of claim 8, wherein said active substance is an anti-inflammatory agent.

11. The method of claim 8, wherein said active substance is cannabidiol.

12. The method of claim 8, wherein said treatment mixture is configured to treat the walls of the digestive tract during traversal of the digestive tract.

13. The method of claim 8, wherein said active substance includes cannabidiol and THC.

14. A method of providing a treatment mixture for treating the digestive tract of a patient, comprising the steps of:
Mixing one or more cannabinoids with a soluble fiber to form the treatment mixture;
packaging the treatment mixture for sale; and
dissolving the treatment mixture in a liquid for consumption by the patient, wherein, said treatment mixture is configured to traverse a substantial portion of the digestive tract of the patient subsequent to said consumption to treat the digestive tract with the active substance along said portion.

15. The method of claim 14, wherein said packaging includes the step of packaging said treatment mixture into individual dose packages.

16. The method of claim 14, wherein said treatment mixture is configured to treat the walls of the digestive tract during traversal of the digestive tract.

17. A method of providing a treatment mixture for treating the digestive tract of a patient, comprising the steps of:
mixing cannabidiol with a soluble fiber to form the treatment mixture;
packaging the treatment mixture into individual dose packages; and
treating the patient by administering an individual dose package to the patient, wherein subsequent to said administering step, said treatment mixture is configured to traverse a substantial portion of the digestive tract of the patient to treat the digestive tract with the active substance along said portion.

18. The method of claim 17, wherein said treatment mixture is configured to treat the walls of the digestive tract during traversal of the digestive tract.

19. The method of claim 17, further comprising the step of mixing THC with the soluble fiber to form the treatment mixture.

20-28. (canceled)

31. The method of claim 14, wherein said one or more cannabinoids includes THC or terpenes derived from marijuana.

32. The method of claim 14, wherein said one or more cannabinoids includes cannabidiol.
